(19) United States
(12) Patent Application Publication
(10) Pub. No.: US 2012/0094369 A1
(43) Pub. Date: Apr. 19, 2012

(54) Test Material, Cassette and Meter for Using the Test Material

(76) Inventors: James Richard Jackson, Hampshire (GB); Richard F. Palmer, Bucks (GB)

(21) Appl. No.: 13/262,555

(22) PCT Filed: Mar. 30, 2010
(86) PCT No.: PCT/GB2010/000614

§ 371 Date: Dec. 12, 2011

(30) Foreign Application Priority Data
Mar. 31, 2009 (GB) 0905552.6

(51) Int. Cl. C12M 1/34

(52) U.S. Cl. 435/287.7

(57) ABSTRACT

A test material for providing an indication of a substance in a sample applied to the test material, the test material comprising: an elongate, flexible, substrate; and a plurality of test sites arranged along the substrate, wherein each test site comprises one or more test reagents, and wherein the one or more test reagents are operable to produce said indication, a cassette for housing such a test material and a test meter.

The present invention relates to test materials, cassettes and test meters and, in particular but not exclusively, to a test material having multiple test sites for measuring the level of glucose in blood samples and a test meter suitable for using such a test material.

A variety of “bio-sensors” are known for testing bodily fluids from organisms such as plants or animals, particularly humans. A typical bio-sensor comprises one or more reagents on an inert substrate. The reagents may, for example, comprise a chemical or a mixture of chemicals provided at test sites. Typically, the reagents are provided in a dried form. Application of a bodily fluid to the test site acts to hydrate the reagents, facilitating a chemical reaction between the reagents and substances present in the bodily fluid. The reagents are typically chosen so that they are effective to provide an indication of a substance of interest in the bodily fluid, for example an indication of the presence of the substance (i.e. a minimum detectable concentration) or a quantitative or semi-quantitative indication of the concentration of the substance. For example, the indication may take the form of a change in colour of the test site, which may be identified visually by the user or, more quantitatively, using a suitable detector device. For example, means may be provided for shining light onto the test site and measuring the reflectivity as a function of wavelength. An alternative approach is to choose the reagents such that the chemical reaction with the bodily fluid will cause an electrochemical reaction detectable using electrodes in the test sites.

A common application of bio-sensors is in the management of type I diabetes. In this context, blood samples may be taken several times a day and a bio-sensor used to measure the glucose concentration in each of the samples. If the measured concentration is too high, insulin may be injected to bring down the level of glucose.

An example of a known test meter for measuring blood glucose is the Ascensia™ DEX®2 manufactured by Bayer. As explained in the user guide for the DEX®2 device (revision 10/02 of the user guide may be found on the Internet at www.bayercarediabetes.com/prodServ/custService/pdf/dex2_eng.pdf), the DEX®2 blood sugar meter uses a disc cartridge containing ten test strips. Each test strip is mounted in a separate cavity of the disc cartridge and is coated with test reagents that are sensitive to glucose. The ten test strips are arranged around the centre of the disc like the spokes of a wheel. When a user wishes to measure their blood sugar level, one of the test strips is ejected from its chamber so that it projects out of the casing of the meter. The user can then deposit a drop of their blood onto the test strip. The meter then measures the glucose level of the blood. Finally, the used test strip is completely ejected from the meter and discarded.

Test meters of the prior art have a number of disadvantages. For example, systems which completely eject the test strips from the meter can be unhygienic. Furthermore, the arrangements often require relatively large test strips and/or bulky systems for storing and delivering the test strips. Furthermore, the test strips of the prior art are produced as individual entities so that manufacture of large numbers of strips is expensive.

It is an object of the present invention to address at least one of the problem discussed above in relation to the prior art.

According to an aspect of the invention, there is provided a test material for providing an indication of the presence of a substance in a sample applied to the test material, the test material comprising: an elongate, flexible, substrate; and a plurality of test sites arranged along the substrate, wherein each test site comprises test reagents, and wherein the test reagents are operable to produce said indication.

Manufacturing a plurality of test sites in this way is less expensive and more efficient than creating a plurality of separate test strips. Moreover, the use of an elongate flexible substrate makes it easier to house the plurality of test sites in an efficient, compact and hygienic manner in comparison with the prior art. The test strips themselves can be made smaller because they do not have to be handled manually by a user.

Preferably, the substrate is housed on a spool in a cassette.

Preferably, the substrate is arranged to have both porous regions and non-porous regions, with the test reagents deposited in the porous regions. The non-porous regions may be arranged so as to surround the porous regions and thereby contain either or both of the reagents and the sample after it has been applied to the test site. Advantageously, the substrate comprising porous regions and non-porous regions may be produced starting from an entirely porous substrate and selectively impregnating the substrate with a non-porous polymer material. This arrangement may lend itself particularly well to efficient, compact housing and/or cost-effective incorporation into delivery apparatus such as cassettes.

Where the reagents are chosen to provide an electrochemically induced indication of the presence of the substance of interest, electrodes may be added to the test material. This may be carried out by screen printing or ink-jet printing, for example.

Red blood cells can interfere with the electrochemistry. It is therefore advantageous to prevent red blood cells from coming into contact with the reagents and/or electrodes. According to an embodiment of the present invention, this can be achieved by selecting a substrate which acts to filter red blood cells and arranging for the reagents and/or electrodes to be spatially separated from the portion of the surface of the substrate onto which the user will apply the sample. For example, the reagents and/or electrodes may be positioned laterally spaced apart from the point of application of the sample, with the system arranged so that the sample is transported, by chromatography, for example, from the point of application to the position of the reagents and/or electrodes. In such an arrangement, the filtering action of the substrate will allow only the components of interest in the blood sample to be transported to the reagents and/or electrodes. Red blood cells will remain substantially at the position of original application of the sample.

Alternatively, the electrodes and/or reagents may be applied to a side of the substrate opposite to the side onto which the sample to be measured is applied. Again, only the components of the blood sample of interest will be transported through to the reagents and/or electrodes. The filtering action of the substrate will prevent any-corresponding movement by the red blood cells and thus prevent their interfering with the electrochemistry.

According to a further aspect of the invention, there is provided a test meter comprising: means for receiving the test material; means for advancing the test material so that an unused test site is presented for use; means for measuring a characteristic of a test site; and means for outputting a measured characteristic.

According to embodiments of the present invention, the test material may readily be made smaller than prior art test materials for a given number of tests. This provides the benefit that an increased number of test sites may be stored within a test meter. For example, a test meter designed to use the test material may readily accommodate up to a hundred test sites. Alternatively or additionally, the test meter may be made more compact than prior art devices.

According to embodiments of the invention, the mechanical path of the test material through the test meter may be simplified, thus enabling the test meter to be manufactured at lower cost.

According to embodiments of the invention, the test material, and cassettes containing the test material, may be manufactured at lower cost than prior art test materials, due to the mechanical simplicity of the test material/cassette.

The invention will be more clearly understood from the following description, given by way of example only, with reference to the accompanying drawings, in which:

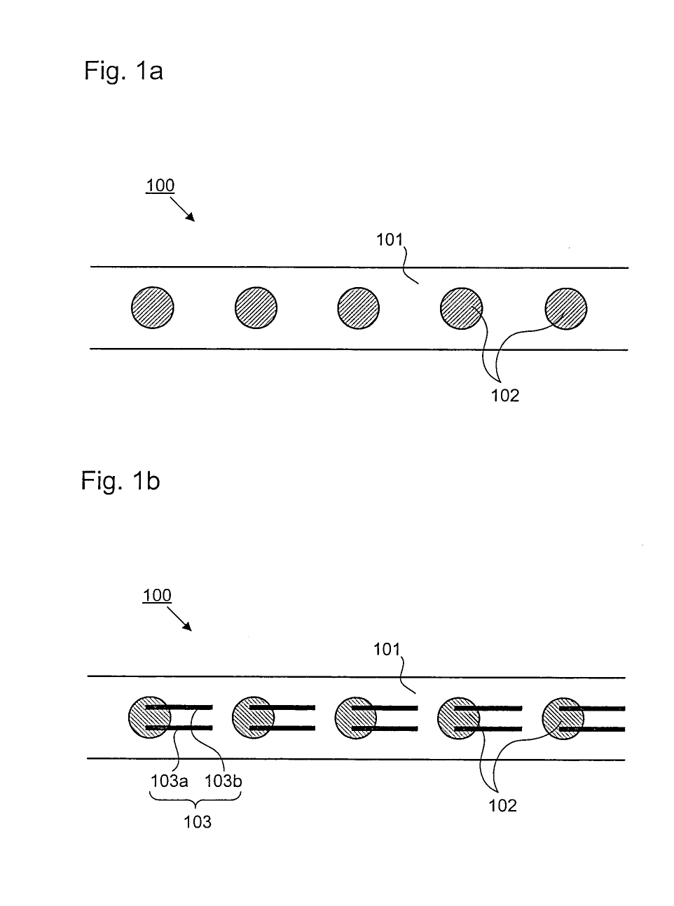

FIG. 1a shows a plan view of the top of a first embodiment of the test material;

FIG. 1b shows a plan view of the underneath of the first embodiment of the test material;

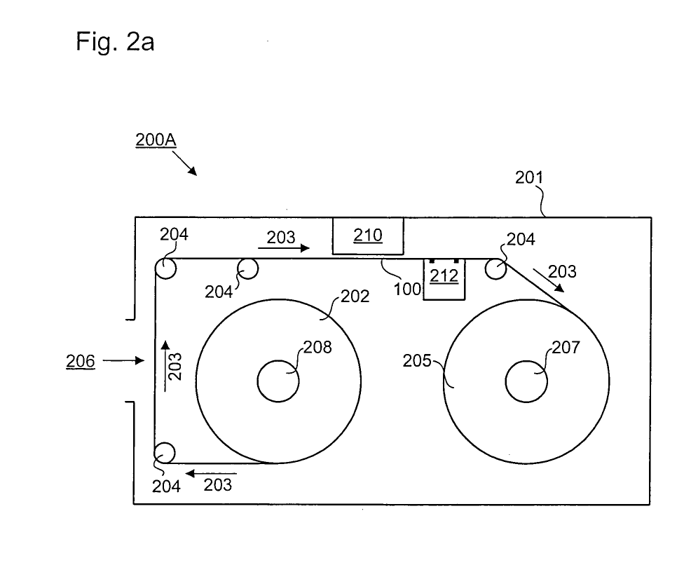

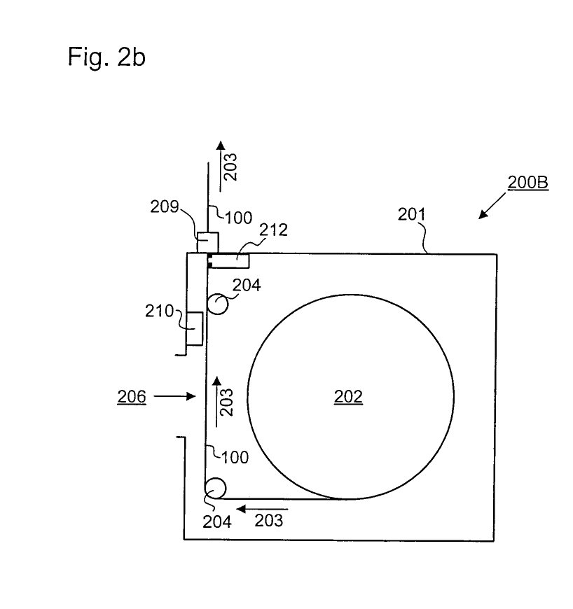

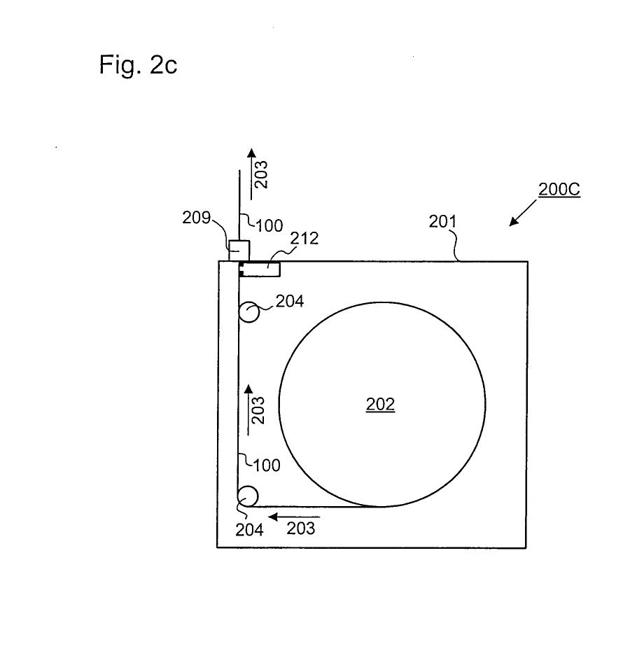

FIG. 2a shows a schematic view of a cassette incorporating the test material of FIGS. 1a and 1b;

FIG. 2b shows a schematic view of an alternative cassette incorporating the test material of FIGS. 1a and 1b;

FIG. 2c shows a schematic view of an alternative cassette incorporating the test material of FIGS. 1a and 1b;

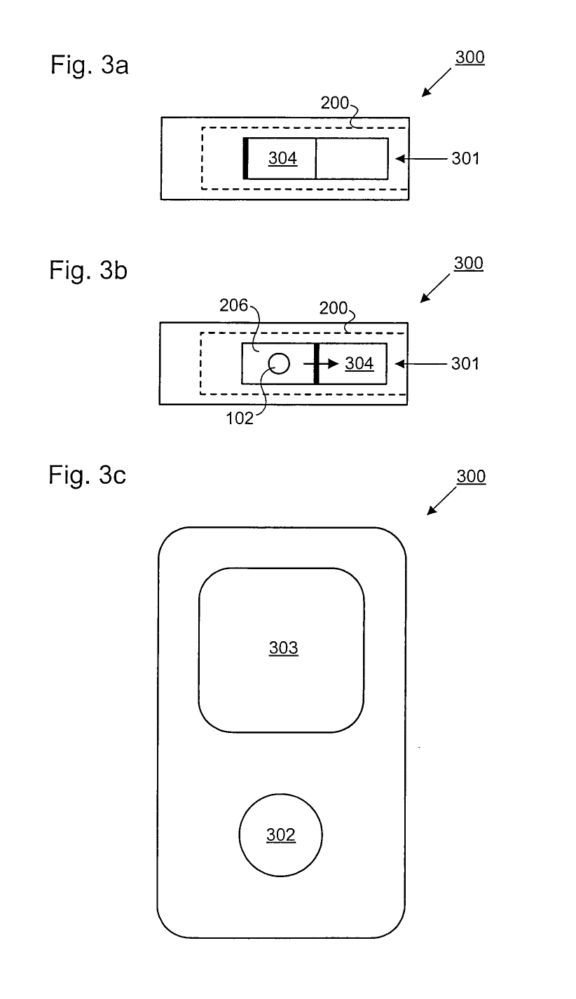

FIG. 3a shows a schematic view of a test meter with a sliding cover in a closed position, suitable for use with a cassette according to an embodiment of the invention;

FIG. 3b shows a schematic view of the test meter with the sliding cover in the open position;

FIG. 3c shows a schematic front view of the test meter showing a display and actuation switch;

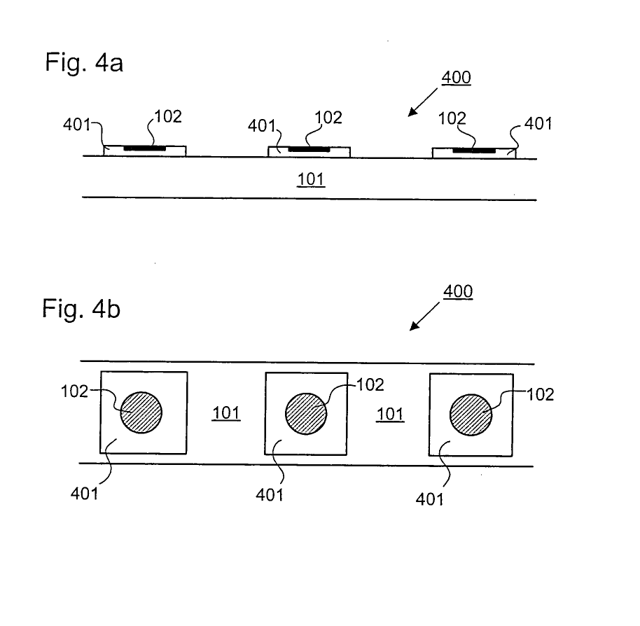

FIG. 4a shows a longitudinal cross-sectional side elevation of a second embodiment of the test material;

FIG. 4b shows a plan view of the top of the test material according to the second embodiment;

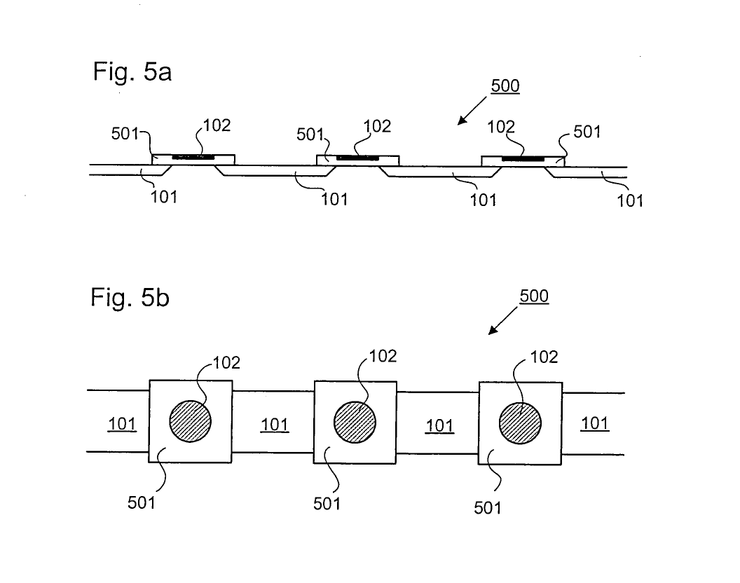

FIG. 5a shows a longitudinal cross-sectional side elevation of a third embodiment of the test material;

FIG. 5b shows a plan view of the top of the test material according to the third embodiment;

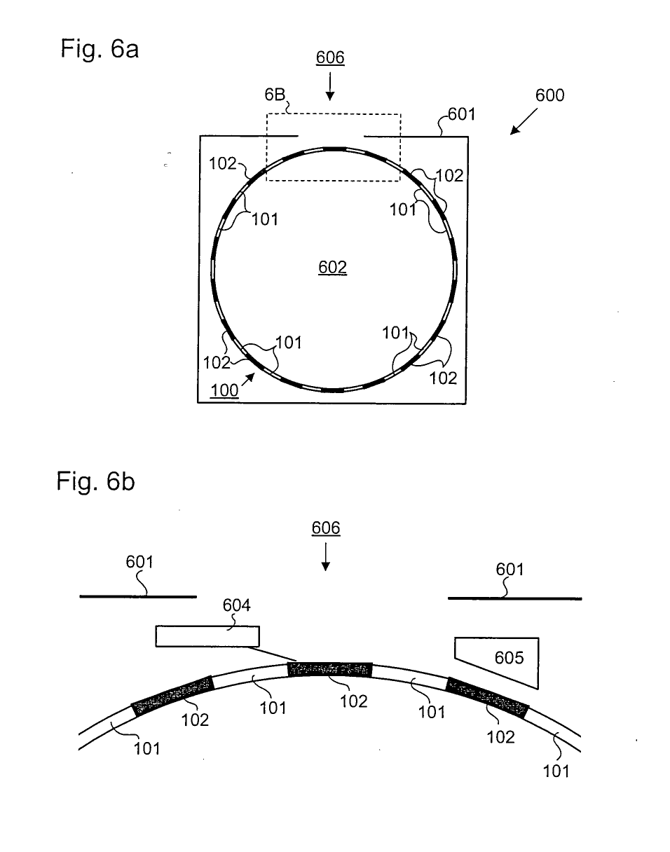

FIG. 6a shows a schematic view of an alternative cassette for incorporating the test material;

FIG. 6b shows a magnified portion of the cassette of FIG. 6a;

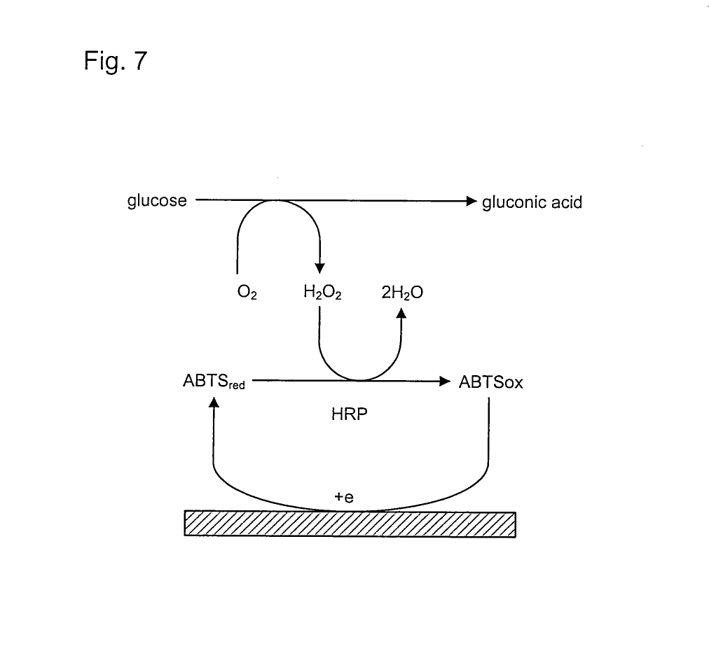

FIG. 7 depicts a reaction scheme for the assay of glucose using glucose oxidase and horseradish peroxidase (IMP) with the electrochemical detection of oxidized ABTS;

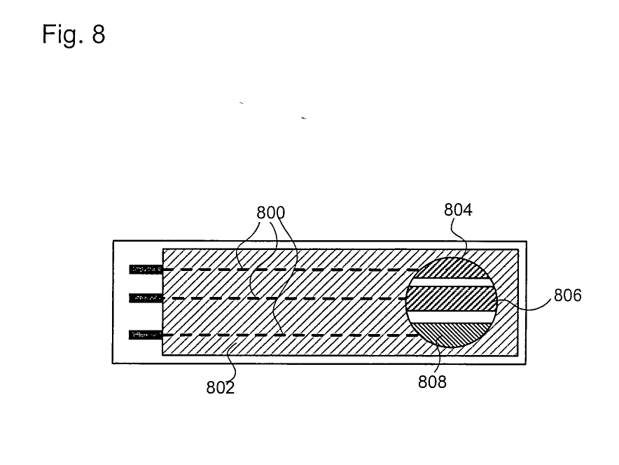

FIG. 8 depicts an example electrode configuration;

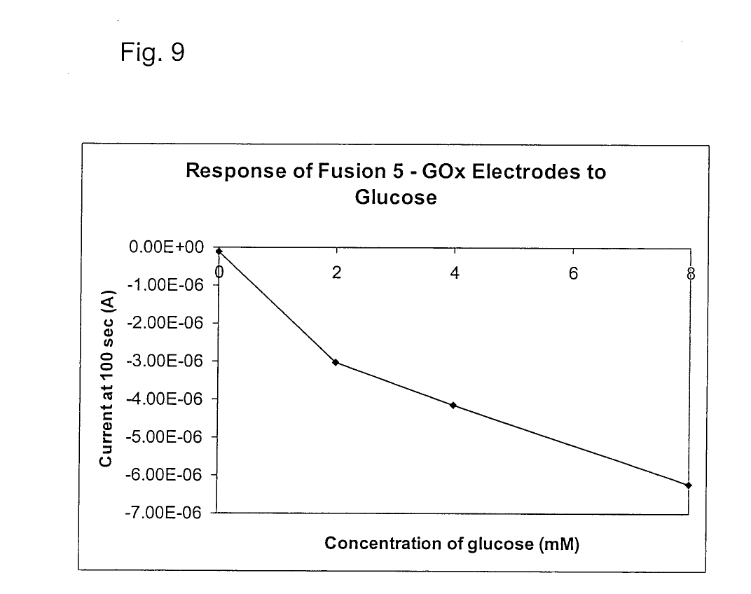

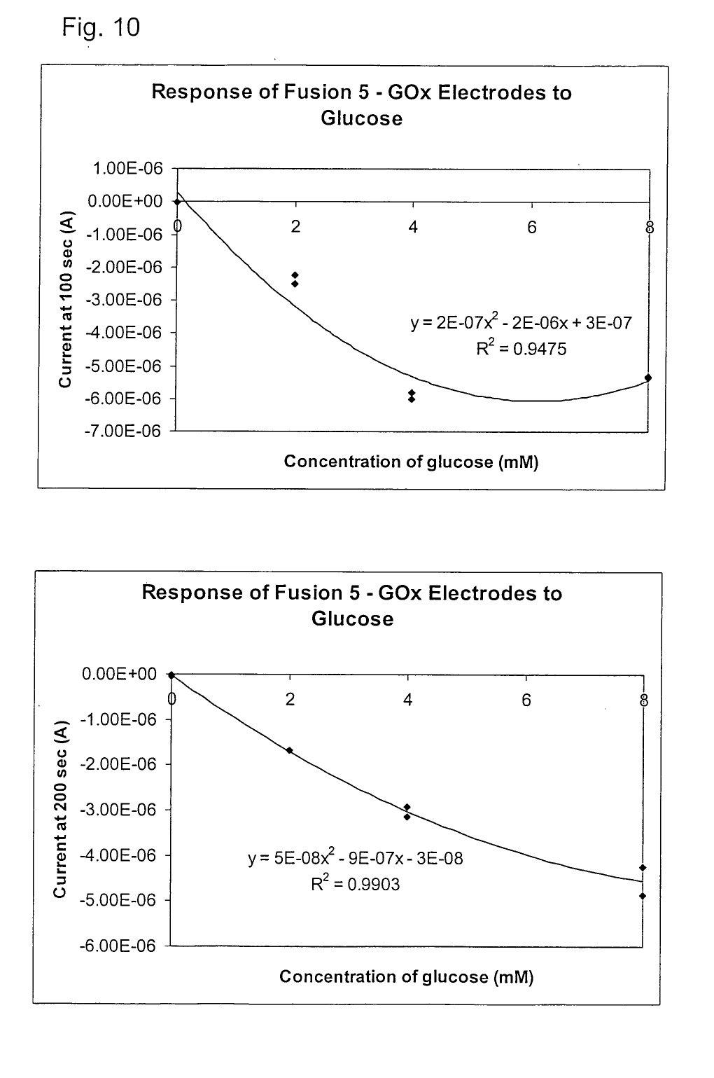

FIGS. 9 and 10 show experimental results of current response of Fusion 5 paper-GOx electrodes to glucose;

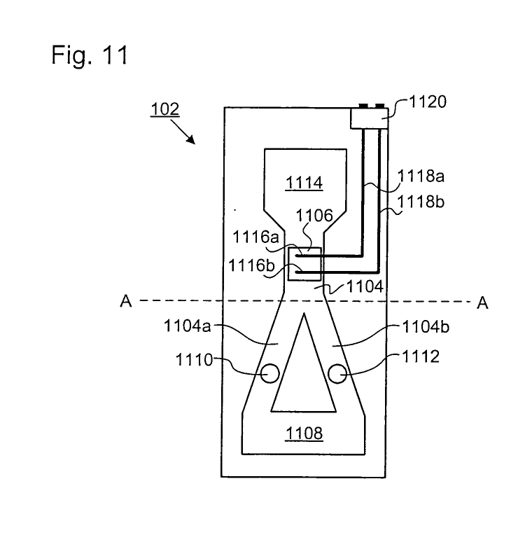

FIG. 11 shows an example configuration of a test site;

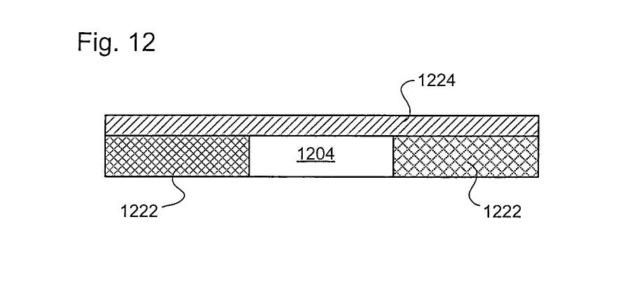

FIG. 12 shows a cross-sectional view along A-A of the test site shown in FIG. 11;

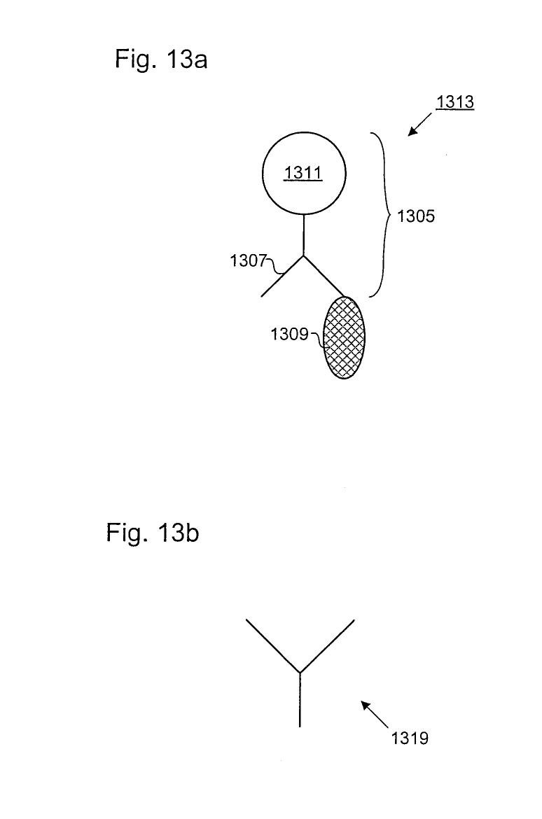

FIG. 13a shows an example complex, comprising a detectable label conjugated to a specific binder that is itself bound to an analyte;

FIG. 13b shows an unbound specific binder for an analyte, immobilized at a testing zone;

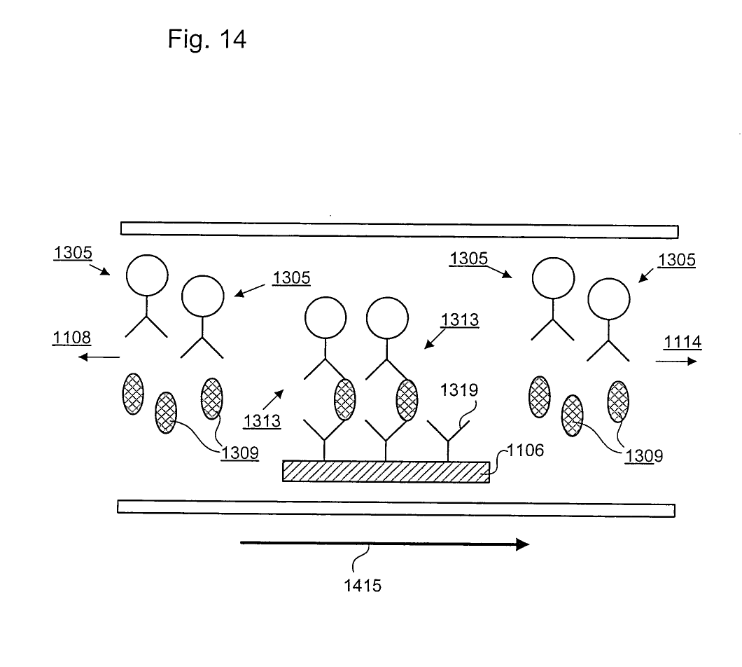

FIG. 14 illustrates a flow of a fluid sample containing a target substance to be detected, together with an added antibody-enzyme conjugate specific to the target substance, through an test site containing an immobilised capture antibody also specific to the target substance; the target substance may bind to either or both of the conjugate and capture antibody;

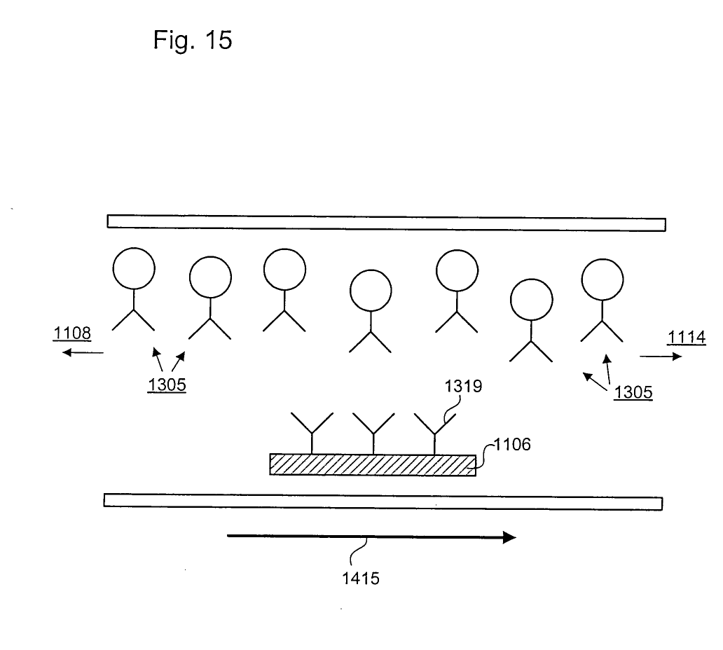

FIG. 15 illustrates a flow of a fluid sample that does not contain a target substance, together with added antibody-enzyme conjugate specific to a target substance, through a test site containing an immobilised capture antibody also specific to a target substance; the absence of a corresponding target substance means no specific binding is possible and therefore the conjugate moves through the testing zone of the test site without becoming bound to a target substance or to a complex of target substance bound to capture antibody; and

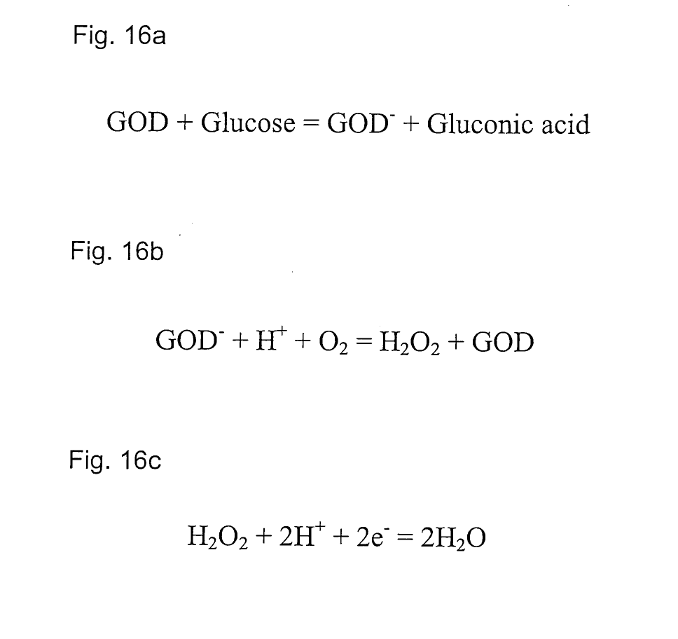

FIGS. 16a to 16c illustrate example reactions for implementing the electrochemical test device by detecting the enzyme present in a conjugate.

FIG. 1a shows a first embodiment of the test material 100. The test material 100 comprises a flexible substrate 101 on which are provided a plurality of discrete test sites 102. In this embodiment the test sites 102 are spaced at a substantially regular distance, for example 2 cm. However, the test sites 102 could be spaced irregularly. Each test site 102 contains one or more reagents that are suitable for providing an indication of the presence and/or concentration of a substance of interest in a sample to be applied to the test site. For example, reagents may be provided that are suitable for reacting with glucose in a sample of blood. The reagents may be provided in dried form on the test sites, which may improve their longevity prior to application of the sample.

The substrate 101 may be sufficiently flexible that it can be wound around a circular former (or any other suitable circular object, or any portion of any other suitable circular object) of 15 cm without damage to the substrate 101. By “without damage”, what is meant is without significant degradation to the ability of the substrate 101 to perform its function in the context of the present invention. Preferably, the substrate 101 may be sufficiently flexible that it can be wound around a circular former (or any other suitable circular object, or any portion of any other suitable circular object) of 10 cm, 5 cm, 1 cm, 0.5 cm or 0.1 cm without damage to the substrate 101.

As shown in FIG. 1b the underside of the substrate 101 may be provided with a plurality of electrodes 103 (also referred to as “electrical contacts”) for carrying out an electrochemical measurement. For example, each test site 102 may be provided with a respective pair of electrodes 103a and 103b.

In the present embodiment, it is intended that a user will apply the sample to be tested to the upper side of the test material (as shown in FIG. 1a), opposite to the electrodes 103. The flexible substrate 101 may in this embodiment be formed from a material, which has a filtering action for blood. In particular, the substrate 101 may be such as to allow all components of the blood sample except red blood cells to move through the substrate material 101 from the top surface to the bottom surface. In this embodiment, it may also be advantageous to locate the dried reagents on the bottom surface of the test material 100, with the electrodes 103. This arrangement thus prevents red blood cells from interfering with the electrochemistry between the electrodes 103a and 103b. A similar effect can be achieved by positioning the dried reagents laterally spaced from the point at which the blood sample is to be deposited on the test material 100.

Alternatively, means may be provided for carrying out an optical test, in which case the electrodes 103 are not necessarily required and may be omitted. Instead, means may be provided for scanning the test site 102 by a wavelength sensitive reflectance photometer, for example. Alternatively, the results of the optical test may be read by eye, for example by comparing the colour change with a suitable calibration chart.

FIG. 2a shows a cassette 200a suitable for housing test materials 100 according to embodiments of the invention, and which may be used in conjunction with the test meter of FIGS. 3a, 3b and 3c (described below). The cassette 200a has a body 201, which may be formed of plastic by injection molding. Unused test material 100 is wound around a supply spool 202. When the test material 100 is to be used, it is passed from the supply spool 202 along a supply path 203, via guide rollers 204, to a take up spool 205. As can be seen, if the take up spool 205 is rotated clockwise, test material 100 will be pulled along the supply path 203 from the supply spool 202, causing the supply spool 202 also to rotate in a clockwise direction. A test aperture 206 in the body 201 provides an aperture through which a sample of bodily fluid (for example blood) may be deposited onto a test site 102 that is positioned facing the test aperture 206. In this embodiment, the body 201 is substantially sealed, other than the test aperture 206, to minimise the possibility of damage to and/or contamination of the test material 100, for example during handling or transportation of the cassette 200.

The take up spool 205 may be provided with a drive socket 207 to enable the take up spool 205 to be driven by an electric motor (not shown). Alternatively or additionally, a mechanism may be provided to allow a user to rotate the take up spool manually. The supply spool 202 may also be provided with a drive socket 208. Although the supply spool 202 will in this embodiment be rotated clockwise upon clockwise rotation of the take up spool 205, the provision of the drive socket 208 allows the test material 100 to be rewound onto the supply spool 202 if required. The drive socket 208 also allows a test meter to verify that the supply spool 202 rotates upon clockwise rotation of the take up spool 205; if the supply spool 202 does not rotate, a test meter can infer that the test material 100 has broken somewhere along the supply path 203.

In alternative embodiments of the cassette 200a, the drive socket 208 is omitted. Similarly, in alternative embodiments of the cassette 200a, the take up spool 205 is not provided with a drive socket 207. Instead, the take up spool 205 may be provided with gear teeth, ridges, or other gripping means (not shown) around its circumference, for example. The gear teeth, ridges, or other gripping means may be arranged to engage with means for electrically rotating the take up spool 205, or may be arranged to protrude slightly from the casing 201 in order to allow a user to rotate the take up spool 205 manually.

Means 210, 212 may be provided within the cassette 200a for facilitating optical and/or electrochemical measurements on test sites 102 after sample deposition. For example, suitable photodetectors 210 and/or electronics 212 may be provided (according to the type of measurement to be made). Alternatively or additionally, suitably positioned apertures may be provided in the cassette 200a to allow access for photodetectors located outside of the cassette. Similarly, all or some of the electronics for carrying out measurements of electrochemistry at the test sites 102 may be located outside of the cassette 200a.

FIG. 2b shows an alternative cassette 200b. In contrast to the arrangement of FIG. 2a, this embodiment comprises only a single spool, the supply spool 202. In this embodiment, the test material is passed from the supply spool 202 along a supply path 203, via guide rollers 204, to an exit aperture/cutoff mechanism 209, where it leaves the body 201 of the cassette 200b. A user can advance the test material 100 in this embodiment simply by gripping and pulling on a portion of the test material protruding outside of the body 201. This pulling action will cause the supply spool 202 to rotate in a clockwise direction. As in the embodiment of FIG. 2a, a test aperture 206 in the body 201 provides an aperture through which bodily fluid may be deposited onto a test site 102 positioned facing the test aperture 206.

In both of the arrangements of FIGS. 2a and 2b, where reagents are chosen to provide an optical indication of the presence of an analyte of interest, the results of the test may be evaluated by a user simply by looking at the test site 102 through the test aperture 206. Alternatively or additionally, means 210 may be provided to measure the change in optical characteristics caused by the reaction of the reagents with the sample of bodily fluid. For example, means 210 may be provided to shine light onto the test site 102 and perform a wavelength sensitive measurement of the reflected light spectrum. This process may be carried out while the test site 102 in question is positioned facing the test aperture 206. Alternatively, the user may advance the test material by a predetermined distance in order to bring the test site 102 to be evaluated to a suitable position within the cassette 200a, 200b, where the optical measurement may be carried out more effectively. For example, the test material may be advanced so that the test site 102 is positioned directly facing a photodetector 210 shown in FIGS. 2a and 2b, or directly facing an aperture through which a photodetector in a test meter into which the cassette is to be inserted may carry out the necessary optical measurements.

Similarly, where the reagents are chosen so as to provide an electrochemically induced indication of the presence of an analyte of interest, the cassette 200b may be arranged to carry out electrical measurements when the test site 102 is facing the test aperture 206 or after it has been advanced to a more convenient position within the cassette body 201. For example, in FIG. 2b, electronics 212 are provided to carry out electrical measurements on the test site after it has been advanced by a predetermined distance from a position facing the test aperture 206 to a position in electrical contact with the electronics 212. Electrical contact with the contacts provided on the rear surface of the test material 100 may be achieved using suitably arranged conductive rollers, for example.

Where it is necessary to advance the test material 100 by a predetermined distance, for example in order to bring the test site into a suitable measurement position, or to pull a used test site outside of the cassette body 201 and bring a new unused test site into an appropriate position opposite the test aperture 206, visible markers may be provided on the test material 100 at suitable spacings.

Once a test site 102 has been used and pulled outside of the body 201, the cutting mechanism 209 may be used to cut the test material 100 at an appropriate point behind the test site 102 to enable it to be released and discarded. The cutting mechanism 209 may consist simply of a serrated edge for manual cutting. Alternatively, means for powered cutting of the test material may be provided.

FIG. 2c shows a further alternative cassette 200c. This arrangement is similar to that depicted in FIG. 2b, except that no test aperture 206 is provided. Instead, a sample of bodily fluid is only applied to a test site 102 when that test site 102 has been pulled outside of the cassette body 201 along the supply path 203. Again, test electronics 212 may be provided to allow electrical measurements to be made subsequently via the electrodes 103 on the rear surface of the test material 100 while the test material is still connected to the cassette 200c (i.e. before the test material 100 is cut using the cutting mechanism 209). Alternatively, the test material 100 may be cut prior to measurement of the electrochemical activity caused by the sample deposition, and inserted into a separate device to carry out the electrical measurements to determine the concentration of the analyte of interest. Similarly, where the reagents are chosen to provide an optical indication, this may be evaluated visually by the user while the test site 102 is still connected to the cassette 200c, or the test site 102 may be cut away from the rest of the test material 100 and the optical indication evaluated elsewhere, for example, using a suitable wavelength-sensitive photodetector.

FIGS. 3a to 3c show an example test meter 300, which may be used with one of the cassette of FIG. 2a, 2b or 2c, for example. FIGS. 3a and 3b are top views of the meter; FIG. 3c is a front view. In this embodiment, the test meter 300 is suitable for measuring the blood glucose level of a diabetic. The test meter 300 is provided with a cassette port 301 that allows a new cassette 200a, 200b or 200c to be inserted into the test meter 300, and also allows a used cassette 200a, 200b or 200c to be removed from the test meter 300. In the present embodiment, the cassette port 301 is built into the side of the meter 300 and is shaped so that when the cassette is inserted, the test aperture 206 is facing upwards. Other arrangements may also be implemented. For example, the cassette port 301 may be configured to allow insertion of the cassette 200a, 200b or 200c from any other direction (top, bottom, front or back).

As can be seen, the test aperture 206 of the cassette is readily accessible by a user of the test meter 300. The test meter 300 may also have an advance button 302 which the user can press to cause actuation of an electric motor (not shown). This motor may drive a spigot (not shown) which engages with a drive socket 207 of the take up spool 205 (where provided) and causes the test material 100 to be advanced along the supply path 203 so that a new test site 102 is presented at the test aperture 206. Alternatively or additionally, a mechanism may be provided for manually driving of the take up spool 205 (as discussed above).

When the test site is presented at the test aperture 206, the user can deposit a sample of bodily fluid, for example blood. In an amperometric bio-sensor, the test meter 300 may apply a constant voltage across the electrodes 103 and measure the resulting electrical current. Alternatively, the test meter 300 may apply an alternating voltage across the electrodes 103 and measure the resulting electrical current. Various other techniques may also be used for evaluating the electrical properties of the liquid located between the electrodes 103. A suitable programmed computer in the meter may be provided to convert the measurements into a concentration of glucose which is displayed on a display 303. Other types of electrochemical bio-sensor could also be used, including for example a potentiometric bio-sensor (which is based on measuring a voltage between the electrodes 103a and 103b).

Alternatively or additionally, the meter 300 may be provided with optical apparatus to carry out optical measurements on the test site 102. For example, means may be provided to measure reflectivity, optionally as a function of wavelength so to yield a quantitative measure of colour change at the test site. This may be carried out via the test aperture 206 or via another suitably positioned aperture in an inserted cassette.

In the embodiment shown, which has the test aperture 206 facing upwards, a sliding cover 304 may be provided to reduce the risk of contamination of the cassette 200 and test sites 102. FIG. 3a shows the sliding cover 304 in the “closed” (i.e. “left” as shown) position, covering the test site 102. FIG. 3b shows the sliding cover in the “open” (i.e. “right” as shown) position, exposing the test site 102 for application of the sample.

The sliding cover 304 may be used to cover the test aperture 206 once a sample of blood has been deposited on to a test site 102 and/or to protect the test aperture 206 prior to application (before the user is ready to deposit a sample). The sliding cover 304 shields the cassette from contact with people other than the primary user, and contamination from dirt and dust.

The cassette port 301 may also be provided with a sliding cover.

In one particular embodiment, the cassette port 301 may be configured so as to have an opening in the same direction as the test aperture 206. In other words, the cassette port 301 may be configured such that the cassette is inserted into the meter in a direction opposite to that which the test aperture 206 will face when inserted (i.e. so that the test aperture 206 is the last part of the cassette that enters the meter 300). In this embodiment, the cassette port 301 and test aperture 206 thus effectively share the same opening and a single sliding cover may be provided that closes both the cassette port 200 and the test aperture 206.

The sliding cover 304 may be linked to a system to activate and deactivate the test meter 300. For example, the meter 300 may be configured such that when the sliding cover 304 is in the open position, the test meter 300 is placed into an “on” state, whereas when the sliding cover 304 is in the closed position the test meter 300 is placed into an “off' state or “standby” state. In alternative embodiments of the test meter 300, the sliding cover 304 is not used to activate/deactivate the test meter 300. In further embodiments of the test meter 300, a sliding cover 304 is not provided. In still further embodiments, means other than a sliding cover 304 are provided to achieve substantially the same functionality. For example, a pivoting cover or a removable cap is provided to close the test aperture 206 and/or cassette port 301.

FIG. 4 shows a second embodiment of the test material 400. FIG. 4a shows a cross section through the test material 400 along the longitudinal axis of the test material 400. FIG. 4b shows a top view of the test material 400.

This test material 400 comprises a flexible substrate 101 and test sites 102 similar to those described above for other embodiments. The test material 400 differs in that the test material 400 comprises “rigid” plates 401 that are attached to the substrate 101. By rigid, it is meant that the plates 401 are stiffer than the flexible substrate 101. The rigid plates 401 are attached to the substrate 101 by a suitable adhesive. An advantage of the test material 400 is that the rigid plates 401 provide mechanical enforcement to the test sites 102.

In this embodiment, each test site 102 is provided with a respective rigid plate 401.

The rigid plates 401 may be formed from non-porous material, similar for example to that used for the test strips of prior art systems such as the Ascensia™ DEX° device manufactured by Bayer. In contrast to such prior art arrangements, however, the rigid plates need not be large enough to facilitate manual handling by a user because such handling is not necessary according to the present embodiment (the rigid plates are supported and conveyed by the flexible substrate on which they are mounted). Thus, the rigid plates according to this embodiment may be made much smaller than prior art test strips having a similar purpose, which facilitates economical and compact housing of a large number of strips.

FIG. 5 shows a third embodiment of the test material 500. The test material 500 is formed of rigid substrate plates 501 similar to the rigid plates 401 of the second embodiment. However, instead of being mounted on a continuous flexible substrate 101, the rigid plates 501 of this embodiment are connected together by discontinous portions of flexible substrate 101.

As shown, the rigid substrate plates 501 have a length, in the direction of the longitudinal axis of the flexible substrate 101, that is comparable with the separation of consecutive test sites 102. In an alternative embodiment, the rigid substrate plates 501 are of a size that is comparable to the size of the test sites 102.

FIGS. 6a and 6b depict an alternative arrangement for a cassette 600 suitable for use with test materials and test meters according to embodiments of the invention. In a similar way to the cassettes 200a, 200b and 200c depicted in FIGS. 2a, 2b and 2c, respectively, the test material 100 according to this embodiment is also wound around the circumference of a spool 602. In contrast to the cassettes 200a, 200b and 200c discussed previously, on the other hand, in the present embodiment all of the test material 100 remains in contact with the spool 602 as the cassette is consumed (i.e. as test sites 102 are used). According to this arrangement, the test material 100 is wrapped around the circumference of the spool 602, for example in a closed loop, so as to form a single layer of test sites. Unlike in previous embodiments, the test material 100 is arranged so as to be fixed relative to the spool; rotation of the spool in either direction does not cause the test material 100 to become unwound or in way to change its spatial relationship with the spool 602; no relative movement occurs between the test sites 102 and the spool 602 during rotation of the spool 602. Each test site 102 may remain adjacent to the same portion of the spool circumference for all angular positions of the spool relative to the cassette housing. A plurality of test sites 102 are arranged around the circumference of the spool facing outwards and angularly separated from each other. The test sites may be brought successively into a position facing a test aperture 606 by rotating the spool 602 (either manually or using an electric motor, for example). According to a variation, a plurality of layers of test material 100 can be provided, each being formed as a closed loop and behaving as above. When all of the test sites 102 on a given layer have been used, the layer may be discarded to reveal a fresh layer underneath.

As shown in the magnified view in FIG. 6b of the broken line delimited region in FIG. 6a, means may also be provided to carry out optical and/or electronic measurements at the test sites 102 after application of a sample. For example, test electronics 604 may carry out electrical measurements while the test site 102 is facing the test aperture 606, for example. Alternatively or additionally, optical measurement means 605 may be provided for carrying out optical measurements of the test site 102 after the test site has been rotated away from the position facing the test aperture 606. Other arrangements are also possible. For example, the electrical measurements could be made after the test site 102 has been moved away from the test aperture 606 and/or the optical measurements could be carried out while the test site 102 is facing the aperture 606.

This embodiment requires few mechanical parts and can be manufactured relatively cheaply and reliably. The arrangement also lends itself to miniaturisation in a cost-effective manner. Arranging a plurality of superposed layers enables compact housing of a larger number of test sites without significantly compromising these fundamental advantages.

The test sites 102 may be configured such that all of the reagents are positioned in the region where the optical or electrochemical indication will take place. This would be the case when using a reaction scheme such as that depicted in FIG. 7, which is suitable for electrochemical detection of the concentration of glucose in a blood sample (further details are given below).

Alternatively, the reagents may be chosen so as to be suitable for a test which relies on the sample being transported (for example by chromatography or capillary action) from a deposition zone to a test zone (where the electrodes are located and the electrochemical indication takes place). In this arrangement, reagents may be located both at the test zone and at an intermediate position between the deposition zone and the test zone. When the sample is applied at the deposition zone in such a system, movement of the sample from the deposition zone to the test zone causes the reagents located between the deposition and test zones to be hydrated and carried to the test zone. An arrangement of this type is shown in FIG. 11.

In FIG. 11, channels 1104, 1104a and 1104b are formed within the test site 102 for containing a flow of a liquid to be tested from a deposition zone 1108, where a user deposits the liquid, to a testing zone 1106 where the liquid is analysed. The flow path is arranged to pass via one or more reagents 1110 and 1112 which are initially localised at positions between the deposition zone 1108 and the testing zone 1106 in channels 1104a and 1104b. The one or more reagents 1110 and 1112 may comprise test reagents that are suitable for the specific determination of the presence of one or more substances in the liquid sample, and may also comprise detection reagents that facilitate detection or measurement of the presence of one or more substances in the sample by an electrochemical means. The test reagents may, for example, comprise an antibody, antigen, receptor or other binder for specifically binding to a target substance for the purpose of its detection or measurement. Examples of possible detection reagents are given below. In the embodiment shown, the channel 1104a contains test reagent and may be referred to as a “test reagent channel”, while the channel 1104b contains detection reagent and may be referred to as a “detection reagent channel”. The test and detection reagent channels 1104a and 1104b are shown as separate channels. In alternative embodiments, a single channel may be provided for both the test and detection reagents. Alternatively, a single channel may be provided for the test reagents only and separate means used for adding the detection reagents (i.e. the detection reagent channel may be omitted). In general, the test and detection reagents will be provided in the form that is localised initially (for example a dry form), but which becomes mobile on arrival of the liquid, such that the reagents are carried along with the liquid. A waste channel 1114 and optional waste sink reservoir may be provided for receiving liquid after it has passed through the testing zone 1106.

Where the substrate 101 is formed of porous paper, the paper may be treated so as to become impermeable to water except at the regions that are destined to become test sites 102. The test reagents may then be deposited onto the porous water permeable regions to form the test sites 102. Where the test chemistry to be used is electrochemical in nature, electrode tracks may first be deposited onto the paper before the paper is treated so as to become impermeable to the water except at the regions that are destined to become test sites 102 and at portions of the electrodes 103 protruding into the test sites (so that they are exposed and can carry out their function). Advantageously, the water impermeable regions of the paper act to insulate and/or protection the portions of the electrode tracks present within these regions. Finally, the test reagents are deposited onto the porous water permeable regions to form the test sites 102.

The flexible substrate 101 of the present invention may be formed by polymer modification of an absorbent or porous material. For example, the process of polymer modification described in WO 09832018 A1 may be used. The absorbent/porous material may initially be provided in generally flat sheet or roll form. The material may typically be either a cellulosic or non-cellulosic based paper and be capable of chromatographically or otherwise transporting a liquid. The absorbent material may be selectively impregnated with a liquid mixture containing polymer or other material which penetrates the absorbent material such that this material becomes selectively impregnated cross-sectionally to a degree of up to 100% and such that the polymer or other impregnating material polymerises or solidifies, rendering the impregnated regions water impermeable. FIG. 12 shows a cross-sectional view of a test material 100 formed in this way through one of the test sites 102. In FIG. 12, regions 1222 correspond to these water impermeable regions and together define the boundaries of a channel 1204 of porous material. Typically, the liquid mixture may be added using a printing technique, for example screen printing or ink jet printing, such that the regions to be impregnated may be predefined and the degree of impregnation across the surface of the absorbent material advantageously controlled to determine the shape and configuration of the impermeable regions, this defining the shape and configuration of the remaining permeable regions. Thus, channels 1204 and sinks or reservoirs may be created and configured selectively. Bio-sensor electrodes, conductive tracks and/or electrical connection interfaces may be printed or otherwise deposited on the test material 100 either prior to or after the selective impregnation process has been implemented, such that these conductive elements can perform their function to facilitate the performance of a test. The impregnated polymer serves to act as an electrical insulator, preventing electrical connection between the electrodes and/or conductive tracks.

The test material 100 thus constructed may further be coated with a water impermeable coating polymer 1224, optionally transparent, as shown in FIG. 12. A coating polymer may be bonded to the substrate 101, optionally by partial penetration of the coating polymer into the porous material such that the coating polymer penetrates from about 30% to about 70% of the thickness of the substrate, so as to define a continuous bibulous compartment using methods such as those described in U.S. Pat. No. 6,573,108 B1. The water impermeable polymer 1224 may also be formed over the conductive tracks to prevent spreading of the tracks, for example where they are formed by printing onto a porous substrate. In this case, the water impermeable polymer may be applied so as to penetrate by 100% of the thickness of the substrate in regions through which the conductive tracks pass, to completely encapsulate the conductive tracks. The polymer may be applied to one or both sides of the substrate in order to achieve this. The polymer may be a UV-curable polymer, for example.

The nature of many potential reagents that may be used is commonly such that they are thermally or otherwise unstable, especially when provided in liquid form. The reagents used may therefore be provided in dry form, typically by the initial application of reagents in liquid form followed by active drying of the reagents or by allowing them to dry naturally. Test materials 100 manufactured in this way will afford thermal stability to the test and detection reagents, facilitating the use of the test material 101 outside of the laboratory or other controlled environment by removing limitations in distribution, storage and handling that the need for refrigeration or cooling would impose. Where the substrate 101 is to be formed from a porous material, a wettable matrix may be used comprised of, for example but not by limitation, cellulose based paper, nitrocellulose membrane, woven or non-woven membranes or other sheet materials, non-cellulosic or synthetic papers.

An example fabrication process for electrodes 103a and 103b is described below, based on screen-printing of the electrodes 103a, 103b onto a porous material comprising glass fibre paper (Whatman “Fusion 5”). The viability of this approach for the fabrication of paper bio-sensors was subsequently evaluated by depositing glucose oxidase on the working electrode and testing the resultant enzyme sensors, using a series of standard glucose solutions. For comparison, electrodes were also printed on cellulose paper.

Before printing, a method for testing Fusion 5 and cellulose paper electrodes was devised. This was based on the electrochemical detection of oxidised 2,2′-azino-bis[3-ethylbenzthiazoline-6-sulfonic acid] (ABTS) generated in the reaction shown in FIG. 7.

In essence, a 5 mg/ml (1270 U/ml) stock solution of glucose oxidase (GOx; Biozyme GO3AC, Biozyme Laboratories, Blaenavon, Gwent, UK.) was made up in 0.1 M sodium phosphate buffer, pH 7.0. A 2 μl aliquot of the enzyme was applied directly to the working electrode 806 of 8 screen-printed Melinex electrodes, each comprising a central carbon working electrode 806, a carbon counter electrode 808 and a silver/silver chloride reference electrode 804 as shown schematically in FIG. 8. Conductive tracks 800 were provided, leading to the reference, working and counter electrodes 804, 806, 808 over the porous material. The conductive tracks were electrically protected by an insulating shroud 802.

The remaining reaction components of the reaction were mixed as follows:

| | |
|---|---|
| 10 mM ABTS | 100 μl; |
| 222 U/ml HRP | 100 μl; |
| 205 mM sodium acetate buffer, pH 5.5 | 500 μl; |
| 1M potassium chloride | 100 μl; |
| reverse osmosis water | 200 μl. |

Glucose solutions were made up at concentrations of 0, 2, 4, 8 and 12 mM in 205 mM sodium acetate buffer, pH5.5 containing 0.1 M potassium chloride.

The electrodes were connected to an AutoLab electrochemical analyser (Ecochemie, Utrecht, Netherlands). A 20 μl sample consisting of equal volumes of glucose and the reaction mix was pipetted over the electrode array and a potential of +150 mV applied. The current over 300 seconds was recorded. The magnitude of the reducing current generated was detected to be proportional to the amount of glucose present, as expected.

The fabrication and testing of screen-printed paper electrodes is now described.

Electrodes were printed on Whatman cellulose paper, grade 1 chr (175 μm thickness) and Whatman Fusion 5 glass fibre paper in a two-stage process using a DEK 248 screen-printer. The Fusion 5 substrate had the following properties:

porosity of 1.65/100 ml/in²;

thickness of 370 μm;

dry tensile strength of 25.2 N/15 mm.

The Whatman cellulose paper was supplied by Cranfield Biotechnology Centre Institute of BioScience & TechnologyCBC.

The electrodes comprised three carbon conducting tracks 800 with a plain carbon working electrode 806, a carbon counter electrode 808 and a silver/silver chloride reference electrode 804. The conducting tracks 800, working electrode 806 and counter electrode 808 were printed as the first layer using Electrodag graphite ink 423SS (Acheson Colloids Co., Plymouth, Devon, UK). The reference electrode was printed in a second layer using Electrodag 10% silver chloride in silver ink 6038SS (Acheson Colloids Co.).

Both layers were printed using DEK stainless steel mesh screens with the following specifications:

325 wire count/inch;

45° mesh orientation;

solvent resistant emulsion, 13μ thickness.

Close inspection of the electrodes showed that there was very little bleeding of the inks into the paper and that the definition of the carbon conducting tracks and all three component electrodes was good.

Initially, the electrodes were printed and tested using glucose oxidase and standard glucose solutions, as described above. However, it became apparent that the sample and reagents rapidly diffuse into the paper when applied to the electrode, resulting in a loss of signal. In order to circumvent this, while also providing both insulation and general protection, electrodes can be coated almost entirely with an insulating shroud 802, comprising for example a polymer or ink, leaving a small area for sample application.

As a first step, a UV curable polymer: Loctite 3106, was evaluated for its efficacy in preventing diffusion of liquid through both Fusion 5 and Whatman cellulose paper. The polymer was painted onto an area of each paper to form an open circle. The polymer was cured by exposure to UV light (UVAPRINT 100 CV) and Loctite 3106 subsequently applied to the rear side of the paper to cover the entire circle. After curing, a 20 μl volume of 3.3 mg/ml Brilliant Blue R-250 dye was applied to the centre of each circle and the degree of sample retention noted. Results indicated that in order to prevent diffusion of the dye into the paper, the Loctite had to be applied to both sides of the Fusion 5 or cellulose paper and the polymer cured for at least 5 minutes after each application.

Loctite polymer 3106 was then painted over the front and rear surfaces of the electrodes, leaving clear a 6 mm area over the electrode array for the sample application and the ends of the conducting tracks for connection to an electrochemical analyser. Each coat of polymer was cured by exposure to a UV light source for 5 minutes. It was necessary to apply a total of two coats of Loctite to each surface to prevent wicking of the test sample away from the site of application.

Initial tests using a multimeter indicated that the application of Loctite to the electrodes did not inhibit conductivity greatly.

Glucose oxidase (GOx) was applied to the working electrode 56 of screen-printed Fusion 5 electrodes, coated with Loctite 3106 as described for the Melinex electrodes (above). Electrodes were subsequently tested with four concentrations of glucose 0, 2, 4 and 8 mM as described above and the current response at 100 and 200 seconds was plotted against glucose concentration. The results from two experiments performed on two different days are shown in FIGS. 9 and 10 (FIG. 9 shows the results from the first experiment and FIG. 10 shows the results from the second experiment).

FIGS. 9 and 10 show that the magnitude of the current response at both 100 and 200 seconds was directly related to the concentration of glucose in the sample. Plotting current at 200 seconds against glucose concentration appeared to generate the optimum calibration curve.

The above-described results demonstrate that screen-printing of electrodes on a cellulose or glass fibre paper substrate is a feasible technique for implementing the electrodes 103a and 103b of the test material 100 of the present invention. An acceptable print definition can be obtained for both of the above-mentioned paper types.

FIGS. 13a to 16c describe how reagents may be chosen in the case where the sample is to be transported (for example by chromatography or capillary action) from a deposition zone to a test zone (where the electrodes are located and the electrochemical indication takes place), for example where the test zone 102 is of the type shown in FIG. 11.

FIG. 13a depicts a complex 1313 that may be used with embodiments of the present invention. Here, an enzyme label 1311 is used, this label being conjugated to a specific binder 1307, such as an antibody specific for a target substance 1309 (“analyte”) to be detected or measured in a sample. In this embodiment, the enzyme-antibody conjugate 1305 would be immobilised or otherwise bound or captured at the testing zone 1106 if the target substance 1309 is present in the sample, by forming a complex 1313 with the conjugate 1305. This could be facilitated by the incorporation of at least a further second specific binder 1319 immobilised or trapped at the testing zone 1106 (FIG. 13b). This further binder 1319 at the testing zone 1106 may be either immobilised directly (for example by chemical bonding or physical adsorption) at the testing zone 1106 or may be captured or otherwise immobilised via a biochemical means (for example via a streptavidin-biotin coupling interaction) or some other means. The presence of bound enzyme label 1311 could be determined electrochemically by coupling a reaction between the enzyme 1311 and one or more appropriate enzyme substrates to an electrode. Such a testing zone 1106 may be referred to as an electrochemical bio-sensor. The electrochemical reaction may optionally be facilitated by the use of an electrochemically active mediator substance present in the testing zone. For example, if the enzyme label were peroxidase, the label could be detected by adding hydrogen peroxide, or a source of hydrogen peroxide, to the testing zone 1106 and coupling or otherwise relating the reaction of the peroxidase and the hydrogen peroxide via a mediator substance, such as TMb or ABTS, to an electrode printed or otherwise present at the testing zone 1106.

Alternative detection reactions using different enzymes and substrates are possible. For example, a preferred embodiment of the present invention might incorporate glucose oxidase as the enzyme and glucose as the substrate and their reaction could be either coupled directly to an electrode which detects hydrogen peroxide produced by the reaction or coupled to a second enzyme, for example peroxidase, in a reaction mixture which consumes hydrogen peroxide, this consumption being measured at an electrode using a mediator substance which participates in the reaction scheme. Alternatively, the glucose oxidase reaction could be coupled to an electrode using a mediator such as ferrocene or a derivative of ferrocene.

More generally, liquid sample travelling from the deposition zone 1108 to the testing zone 1106 will mobilize a test reagent 1110 in a test reagent channel 1104a, forming complex 1313 if analyte 1309 is present. Complex 1313 will be carried to the test zone 1106 where it may be immobilized by binding to a specific binder 1319, thereby changing the electrical properties of the liquid between the electrodes 1116a and 1116b in the testing zone 1106 due to the presence of the detectable label 1311 and/or detection reagents. The electrodes 1116a and 1116b can be used to measure the change in electrical properties, and the concentration or simply the presence of the analyte in the sample under test can be deduced from the measurements. The test system may be arranged, for example, such that the degree of binding at the testing zone 1106 is proportional to the concentration of analyte 1309 in the sample.

FIG. 13b illustrates an example where no specific binding of conjugate 1305 has occurred at the testing zone 1106 and the binding sites of the specific binder 1319 immobilised at the testing zone 1106 are not occupied by analyte 1309 bound to conjugate 1305. In some instances, the immobilised specific binder 1319 may be bound to analyte 1309 that is not bound to conjugate 1305 (not shown). In instances where there is no conjugate 1305 specifically bound at the testing zone 1106, no useful electrochemical signal may be detected. Under normal test conditions, it is possible that conjugate 1305 may become bound non-specifically at the testing zone 1106 (not shown). In an optimised test system, such non-specific binding may occur to a limited extent that would not give rise to an incorrect test result and it is possible optionally to include a negative control means where non-specific binding may be estimated and corrected for when a test result is determined.

FIG. 14 illustrates the process of a liquid sample flowing from the deposition zone 1108 to the sample zone 1106 and sink 1114. The liquid flow within the channels 1104, 1104A and 1104B is indicated by an arrow 1415. Mobilized conjugate 1305 and analyte 1309 are present upstream of the testing zone 1106. At the testing zone 1106, the conjugate 1305 can become bound to the specific binder 1319 immobilized in the testing zone 1106 if it has first become bound to the analyte 1309. Conjugate 1305 that does not bind at the testing zone 1106 may be washed from the testing zone 1106 and into the waste channel 1114. Some of the conjugate 1305 washed from testing zone 1106 may be bound to analyte 1309 from the sample only (not shown).

FIG. 15 illustrates the nature of the absence of binding of test reagents at the testing zone 1106, which gives rise to no useful electrochemical signal being detectable if the test system is of the commonly used “sandwich” type. Lack of binding may result from there being no analyte in a sample or there being a relatively low concentration of analyte in a sample. In a balanced test system the amount of unbound test reagents will increase proportionally as the concentration of analyte in a sample decreases.

FIGS. 16a, 16b and 16c illustrate by way of example a biochemical reaction that could be used as the basis for electrochemical detection of an analyte. In this example, the conjugate 5 comprises the enzyme glucose oxidase (GOD) with glucose as a substrate used in the detection reaction. The GOD converts the glucose to gluconic acid and in the process becomes reduced (GOD−) (FIG. 16a). The GOD− reacts with hydrogen ions (H+) and oxygen (O2) present in the reaction mixture and hydrogen peroxide (H2O2) is produced (FIG. 16b), which may subsequently be detected electrochemically by virtue of a reaction at an electrode in which the hydrogen peroxide is caused to react with hydrogen ions (H+) and electrons (e−) to produce water (H2O2) (FIG. 16c). The electrons participating in the overall reaction scheme interact with the electrode system present at a testing zone 1106 to produce a detectable electrical signal, which may be measured amperometrically for example.

Any of the test materials described in this application can be used with any of the cassettes described in this application, which can in turn be used with any of the test meters described in this application.

What we claim is:

1. A test material for providing an indication of a substance in a sample applied to the test material, the test material comprising:
an elongate, flexible, substrate; and
a plurality of test sites arranged along the substrate, wherein each test site comprises one or more test reagents, and wherein the one or more test reagents are operable to produce said indication.

2. A test material according to claim 1, wherein the substrate comprises a wettable matrix comprised of one of the following: cellulose based paper, nitrocellulose membrane, woven membranes, non-woven membranes, non-cellulosic paper, synthetic paper, another absorbent material, another porous material, another fibrous material.

3. A test material according to claim 1, wherein the one or more test reagents are operable to produce an optical indication of the substance.

4. A test material according to claim 1, wherein the one or more test reagents are operable to produce an electrical indication of the substance.

5. A test material according to claim 4, comprising electrodes for measuring an electrical characteristic of the test sites.

6. A test material according to claim 5, further comprising a water-impermeable polymer impregnated into the substrate so as to at least partially encapsulate at least a portion of said electrodes.

7. A test material according to claim 5, wherein said electrodes are formed by a printing technique onto said substrate.

8. A test material according to claim 5, wherein each of said test sites comprises:
at least one channel for transporting a liquid to be tested from a deposition zone, where said sample can be applied, to a testing zone, the testing zone being spaced apart from said deposition zone.

9. A test material according to claim 8, wherein said at least one channel comprises a porous material capable of transporting said liquid chromatographically.

10. A test material according to claim 1, wherein said substrate comprises porous regions and non-porous regions, at least one of said test sites being provided in one of said porous regions.

11. A test material according to claim 10, wherein said non-porous regions are formed by impregnation of a non-porous polymer into the substrate.

12. A test material according to claim 1, comprising a plurality of non-porous plates, wherein the plates are stiffer than the substrate, and wherein at least one of said test sites is located on one of said plates.

13. A test material according to claim 12, wherein the plates are attached to the substrate by adhesive.

14. A test material according to claim 1, wherein the one or more test reagents are operable to provide an indication of the concentration of glucose in a sample of blood.

15. A test material according to claim 1, wherein the test material is sufficiently flexible to be wound around a circular former of 15 cm diameter without damage to the test material.

16. A test material according to claim 1, wherein said substrate acts as a filter for red blood cells.

17. A cassette comprising:
a test material according to a claims 1; and
a body for housing said test material.

18. A cassette according to claim 17, further comprising:
a test aperture through which a user can apply said sample to a test site on said test material and means for conveying unused test sites to a position facing said test aperture.

19. A cassette according to claim 18, configured such that, where a test site on the test material comprises electrodes for detecting an electrochemical indication, these electrodes are positioned on a side of said test material facing away from said test aperture when that test site is positioned facing said test aperture.

20. A cassette according to claim 17, further comprising a supply spool for storing and supplying said test material.

21. A cassette according to claim 20, further comprising a take up spool for receiving test material supplied by said supply spool.

22. A cassette according to claim 20, configured such that each of said test sites will remain in contact with said supply spool, adjacent to the same circumferential portion of said supply spool, for all angles of rotation of said supply spool relative to said body.

23. A test meter comprising:
means for receiving a test material according to claim 1;
means for advancing the test material so that an unused test site is presented for use;
means for measuring a characteristic of a test site; and
means for outputting a measured characteristic.

24. A test meter according to claim 23, wherein the means for outputting comprises a display.

25. A test meter according to claim 23, wherein the means for receiving a test material comprises means for receiving a cassette, the cassette comprising:
a test material for providing an indication of a substance in a sample applied to the test material, the test material comprising: an elongate, flexible, substrate; and a plurality of test sites arranged along the substrate, wherein each test site comprises one or more test reagents, and wherein the one or more test reagents are operable to produce said indication; and
a body for housing said test material.

26. A test meter according to claim 23, comprising a moveable shutter for selectively exposing and covering a presented test site.

27. A test meter according to claim 26, comprising means for detecting the position of the moveable shutter and for controlling the test meter on the basis of the position of the moveable shutter.

28. A test material, a cassette and a test meter arranged to operate substantially as hereinbefore described with reference to and/or as illustrated in the accompanying figures.
